(19) United States
(12) Patent Application Publication
(10) Pub. No.: US 2007/0297983 A1
(43) Pub. Date: Dec. 27, 2007

(54) Inhibition of breast carcinoma stem cell growth and metastasis

(76) Inventors: Soldano Ferrone, Buffalo, NY (US); Xinhui Wang, Williamsville, NY (US); Tim Clay, Cary, NC (US); Kim H. Lyerly, Chapel Hill, NC (US); Michael A. Morse, Raleigh, NC (US); Gay Devi, Morrisville, NC (US); Takuya Osada, Chapel Hill, NC (US)

Correspondence Address: HODGSON RUSS LLP;THE GUARANTY BUILDING, 140 PEARL STREET, SUITE 100, BUFFALO, NY, 14202-4040, US

(21) Appl. No.: 11/724,884

(22) Filed: Mar. 16, 2007

Related U.S. Application Data
(60) Provisional application No. 60/783,091, filed on Mar. 16, 2006.

(51) Int. Cl. A61K 39/395; A61K 49/00; A61P 35/00; G01N 33/574

(52) U.S. Cl. 424/9.2; 424/130.1; 424/156.1; 435/7.1

(57) ABSTRACT

Disclosed is a method for inhibiting the growth of breast carcinoma stem cells. that express High Molecular Weight -Melanoma Associated Antigen (HMW-MAA). The method comprises administering to an individual a composition comprising an antibody reactive with HMW-MAA or a fragment of such an antibody in an amount effective to inhibit the growth of the breast carcinoma cells. Also provided are methods for inhibiting metastasis of breast carcinomas and methods for identifying HMW-MAA+ breast cancer stem cells.

This application claims priority to U.S. Provisional Application Ser. No. 60/783,091, filed Mar. 16, 2006, the disclosure of which is incorporated herein by reference.

### FIELD OF THE INVENTION

The present invention relates generally to the field of cancer and more particularly to inhibiting the growth of breast carcinoma stem cells.

### BACKGROUND OF THE INVENTION

Cancers of epithelial origin are responsible for the majority of cancer-related deaths from incurable metastatic disease. The cancer stem cell hypothesis (Reya et al., (2001) Nature 414:105) proposes that certain tumors originate from and persist due to mutations in tissue stem cells that result in unregulated, immortal proliferation, and in this state are referred to as cancer stem cells (CSC). It has long been recognized that only a very small percentage of cells in a tumor are capable of immortal growth (approximately 1/1000 to 1/5000 cells in lung tumors and 1/1,000,000 in leukemia cells (Reya, (2001); Dick, J. E. (2003) Proc Natl Acad Sci 100:3547; Marx, J. (2003) Science 301:1308.). There is now very good evidence that a number of cancers, including breast Gudjonsson, et al. (2002) Genes Dev 16:693; Al-Hajj, et al. Proc Natl Acad Sci 100:3983; Dontu, G., et al., (2004) Breast Cancer Res 6:R605; Ponti, D., (2005) Cancer Res 65:5506); colon (Kim, et al. (2005) Cell 121:823), ovarian (Bapat, et al. (2005) Cancer Res 65:3025), lung (Kim, et al. (2005) Cell 121:823, and prostate cancers (Schalken, (2003) Urology 62:11.), leukemia (Dick, J. E. (2003), glioma (Kondo, et al. (2004) Proc Natl Acad Sci 101:781; Singh, S. K., et al. (2004) Nature 432:396), retinoblastoma (Reedijk, M., S. et al. (2005) Cancer Res 65:8530) and hepatocellular carcinoma (Rosner, A., K. et al. (2002) Am J Pathol 161:1087) proliferate from cancer stem cells. Cancer stem cells have also been isolated from established tumor cell lines (Gudjonsson, et al. (2002) Genes Dev 16:693; Ponti, D., (2005) Cancer Res 65:5506; Kondo, et al. (2004) Proc Natl Acad Sci 101:781), and retain the same phenotype as the tumors from which they were originally isolated. Evidence in several types of cancer shows that pathways prominent in normal stem cell function, notably Wnt, Notch, Ssh (sonic hedgehog), XIAP (X-linked inhibitor of apoptosis protein) become ‘dysregulated’ in cancer stem cells (CSC) (Reya et al., (2001) Nature 414:105; Dontu, G., et al., (2004) Breast Cancer Res 6:R605; Rosner, A., K. et al. (2002) Am J Pathol 161:1087; Reya, T., et al. (2005) Nature 434:843; Li, Y., B. et al. Proc Natl Acad Sci 100: 15853; Yang, L., Z. et al. (2003) Cancer Res 63:6815; Liu, S., et al. (2005) Breast Cancer Res 7:86; Mikaelian, I., et al. (2004) Breast Cancer Res 6:R668).

Screening mammography is highly effective in identifying breast cancer in women, and it is estimated that in 2005, more than 211,000 new cases of invasive breast cancer and approximately 58,000 new cases of in situ breast cancer will be identified in the U.S. (Society, A. C. Breast cancer Facts and Figures American Cancer Society 2005). Breast cancer is the leading cause of cancer death in women (Sasco, A. J. (2003) Horm Res 60 Suppl 3:50.) with more than 40,000 deaths annually (Society, A. C. Breast cancer Facts and Figures American Cancer Society 2005) due to recurrence of local and distant metastasis. Breast cancer recurrence has been linked to the presence of systemic micrometastases. The therapeutic resources are limited, since Herceptin which, in combination with radiotherapy and chemotherapy reduces the rate of recurrences (Bapat, S. A., et al. (2005) Cancer Res 65:3025) can be applied to only 30% of breast cancer patients (HER2 positive).

The high rates of recurrence and metastasis, even following surgery, chemotherapy, radiation, targeted small molecule and antibody therapies—all of which shrink tumors but do not eliminate immortal tumor cells—underscore the need to identify new therapeutic strategies that specifically target and kill cancer stem cells in order to eliminate recurrence and metastatic disease. Therefore, there is an ongoing need for understanding the neoplastic changes that occur uniquely in cancer stem cells can lead to an understanding of how CSC tumors form, how they proliferate, how they evade standard treatments, and for the development of therapies that target cancer stem cells.

### SUMMARY OF THE INVENTION

The present invention provides a method for inhibiting the growth of breast carcinoma stem cells. The breast carcinoma stem cells express High Molecular Weight—Melanoma Associated Antigen (HMW-MAA). The method comprises administering to an individual a composition comprising an antibody reactive with HMW-MAA in an amount effective to inhibit the growth of the breast carcinoma stem cells.

In another embodiment, a method is provided for inhibiting metastasis of a breast carcinoma where the breast carcinoma comprises HMW-MAA+ breast carcinoma stem cells. The method comprises administering to the individual a composition comprising an amount of an antibody reactive with HMW-MAA effective to inhibit metastasis of the breast carcinoma.

In another embodiment, a method is provided for detection of HMW-MAA+ breast carcinoma stem cells. The method comprises administering to an individual, or contacting a biological sample obtained from the individual with, a combination of antibodies, where the combination of antibodies comprises an antibody directed HMW-MAA and at least one antibody directed to a breast cancer stem cell marker. Detecting the binding of both the HMW-MAA antibody and the at least one breast cancer stem cell marker determines the presence of an HMA-MAA+ breast carcinoma stem cell.

In particular embodiments, the antibody employed in practicing the invention can be the monoclonal antibody designated 225.28 and/or the monoclonal antibody designated 763.74.

### BRIEF DESCRIPTION OF THE FIGURES

FIGS. 1A and 1B present a graphical representation of data obtained by fluorescence activated cell sorting (FACS) of HMW-MAA expression by a subpopulation of breast carcinoma stem cells.

FIG. 2 is a photographic representation of a Western blot analysis of HMW-MAA expressed by MDA-MB-435 cells.

FIGS. 3A and 3B are graphical representations of data obtained from FACS separation of MDA-MB-435s cells stained with antibodies to breast carcinoma stem cell markers.

FIG. 4 is a graphical depiction of results obtained from inhibition by HMW-MAA-specific mAb 763.74 and 225.28 of human breast cancer cell MDA-MB-435s lung metastases in SCID mice.

FIG. 5 is a graphical depiction of results obtained from inhibition of post-surgery lung metastasis of human breast carcinoma stem cells by use of mAb 225.28

### DETAILED DESCRIPTION OF THE INVENTION

The present invention is related to the discovery that HMW-MAA is present on breast carcinoma stem cells. The invention provides a method of inhibiting the growth of breast carcinomas which comprise HMW-MAA+ breast carcinoma stem cells. The method comprises administering to an individual a composition comprising an antibody reactive with HMW-MAA in an amount effective to inhibit the growth of the breast carcinoma stem cells.

Also provided is a method for inhibiting metastasis of a breast carcinoma in an individual, wherein the breast carcinoma comprises HMW-MAA+ breast carcinoma stem cells. The method comprises administering to the individual an amount of an antibody reactive with HMW-MAA effective to inhibit the metastasis.

In another embodiment, a method is provided for detection of HMW-MAA+ breast carcinoma stem cells by administering a combination of antibodies to an individual or a biological sample obtained from the individual. The combination of antibodies comprises an antibody directed to HMW-MAA and at least one antibody directed to a breast cancer stem cell marker. Detection of the binding of the HMW-MAA antibody and the antibody to the breast cancer stem cell marker determines the presence of an HMA-MAA+ breast carcinoma stem cell.

With respect to HMW-MAA, it is a highly glycosylated integral membrane chondroitin sulfate. It consists of an N-linked 280 kDa glycoprotein component and a 450 kDa chondroitin sulfate proteoglycan component. The two components share the same core protein. Through the use of mouse and human monoclonal antibodies, a number of its antigenic determinants have been identified. They display a heterogeneous expression on melanoma cells line and in melanoma lesions. HMW-MAA plays a role in the growth and metastatic potential of melanoma cells, and while one report observed HMW-MAA expression on breast carcinoma cells (Dell'Erba et al., (2001) Anticancer Res. March-April; 21(2A):925-30), the present discovery that HMW-MAA is expressed on breast carcinoma stem cells is unique in that there is presently no evidence that antigens expressed by tumor cells are also expressed by cancer stem cells. In connection with this finding, we demonstrate that substantial percentages of breast carcinoma stem cells obtained from pleural effusions of breast cancer patients include breast carcinoma stem cells that express HMW-MAA. Further, we demonstrate that the method of the invention can be used to inhibit metastasis of carcinomas formed in an animal model inoculated with human breast carcinoma stem cells that we have determined express HMW-MAA. Further still, we demonstrate that post-resection reoccurrence of carcinomas formed from human breast cancer stem cells that express HMW-MAA can be effectively inhibited using the method of the invention. Thus, the method is expected to provide a unique therapy for breast cancer patients by targeting breast carcinoma stem cells that express HMW-MAA.

Breast carcinoma stem cells are considered those breast carcinoma cells that express CD44 (“CD44+”), but do not express CD24 (“CD24−”) or express low amounts of CD24 (“CD24lo”) relative to normal cells or non-stem cells. ESA is also known to be a marker of breast carcinoma stem cells, while B38.1 is known to be breast cancer cell. Non-stem cells are considered those which express one or more of CD2, CD3, CD10, CD16, CD18, CD31, CD45 CD64, and CD140b. Accordingly, cells expressing any of CD2, CD3, CD10, CD16, CD18, CD31, CD45, CD64 or CD140b are not considered breast carcinoma stem cells. It will be recognized by those skilled in the art that other markers for identifying breast carcinoma stem cells may be known or identified hereafter and may be used in identifying breast carcinoma stem cells in connection with the present invention.

The aforementioned markers can be used to identify breast carcinoma stem cells using conventional methods such as immunohisotchenistry or cell sorting. In one embodiment, breast carcinoma stem cells can be identified essentially using the cell sorting methods and markers described by Al-Hajj, et al. (PNAS (2003) Vol. 100, pp 3984-3983). The present invention provides an adaptation of this method such that breast carcinoma stem cells that express HMW-MAA can be identified using anti-HMW-MAA antibodies.

In one embodiment, identification of breast carcinoma stem cells can be performed by flow cytometry using standard cell sorting procedures. For example, cells obtained from patient effusions or biopsies using conventional techniques may be processed by first ficolling the fluid (typically 500 ML-2L) to remove debris and red blood cell contamination. Gating can also be carried out (for example, with antibody directed to CD45) to distinguish over blood cells. Flow cytometric staining for breast carcinoma stem cell phenotypic analysis can identify “lineage negative” cells (negative for CD2, 3,10,16,18,31,45,64,140b, for example, using PE labeled antibodies). For FACS analysis, CD44+-FITC labeled antibody/CD24lo PerCP labeled antibody (all antibodies from BD/Pharmingen, San Jose, Calif.) can be used to assay cells from human breast cancer patient pleural effusions. Cell sorting from malignant effusions can optionally first use anti-PE coated beads to deplete the lineage marker positive cells to greatly reduce the number of non-carcinoma stem cells and thereby reduce the cell sorting time.

In another embodiment, a patient sample can be assessed for the presence and percentage of various cell populations by flow cytometry sorting of ESA⁺CD44⁺CD24^{-/low} cells, as per Al-Hajj et al. In combination or in series with this staining, the ESA⁺CD44⁺CD24^{-/flow} cells can be stained with an anti-HMW-MAA antibody to identify breast carcinoma stem cells that express HMW-MAA. Optionally, staining can be carried out with more than one antibody directed toward HMW-MAA which are each directed to different epitopes of HMW-MAA. Non-limiting examples of monoclonal antibodies suitable for use in this method include the anti-HMW-MAA antibodies designated 225.28 and/or the monoclonal antibody designated 763.74.

HMW-MAA antibodies of the invention can be used be used for a variety of diagnostic assays, imaging methodologies, and therapeutic methods in the management of breast cancer. For example, efficacy of the present method in inhibiting the growth of, or eliminating breast carcinoma stem cells in an individual could be ascertained by analysis of samples obtained from the individual before and after treatment, such as by analysis of pre- and post-treatment biopsies, immunohistochemical analysis, or cell sorting analysis to determine the presence of breast carcinoma stem cells that express HMW-MAA.

Anti-HMW-MAA antibodies can be conjugated to various moieties for diagnostic or therapeutic applications related to HMW-MAA+ breast carcinoma stem cells. For example, anti-HMW-MAA antibodies may be conjugated to a therapeutic agent to enable localization of the therapeutic agent to breast carcinoma stem cells which express HMW-MAA. Examples of suitable therapeutic agents include, but are not limited to, an anti-tumor drug, a toxin, a radioactive agent, a cytokine, a second antibody or an enzyme. Examples of cytotoxic agents include, but are not limited to ricin, ricin A-chain, doxorubicin, daunorubicin, taxol, ethiduim bromide, mitomycin, and the like.

In another embodiment, the anti-HMW-MAA antibodies may be conjugated to a radioactive agent. A variety of radioactive isotopes are available for conjugating to mAbs such that breast carcinoma stem cells that express HMW-MAA may be imaged or selectively destroyed. For selective destruction of cells, the antibodies may be conjugated to a highly radioactive atom, such as In¹¹¹, At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu.

When the antibody conjugates are used for identifying breast carcinoma stem cells which express HMW-MAA, the antibody conjugates may comprise any suitable detectable markers which include, but are not limited to, a radioisotope, a fluorescent compound, a bioluminescent compound, chemiluminescent compound, a metal chelator or an enzyme. For example, certain radioisotopes can be used for scintigraphic studies, such as Tc^{99m} (metastable technetium-99), I¹²³, or as spin labeled atoms for nuclear magnetic resonance (NMR) imaging (also known as magnetic resonance imaging, or “MRI”), such as I¹²³, I¹³¹, I¹²⁴, F¹⁹, C¹³, N¹⁵, O¹⁷ or Gadlinium (III) or Manganese (II). Such labels may be incorporated in into the antibodies in known ways. “Monoclonal Antibodies in Immunoscintigraphy” (Chatal, CRC Press 1989) describes suitable methods in detail.

In addition to the antibodies disclosed here, other antibodies to HMW-MAA can also be produced. The methods for producing monoclonal and polyclonal antisera are well known in the art. The antibodies or fragments may also be produced by recombinant means. Alternatively, fully human monoclonal antibodies can also be produced by methods such as phage display and transgenic methods (Vaughan et al., 1998, Nature Biotechnology 16: 535-539). For example, fully human anti-HMW-MA monoclonal antibodies may be generated using large human Ig gene combinatorial libraries (i.e., phage display); (Griffiths and Hoogenboom, Building an in vitro immune system: human i antibodies from phage display libraries. In: Protein Engineering of Antibody Molecules for Prophylactic and Therapeutic Applications in Man. Clark, M. (Ed.). Nottingham Academic, pp 45-64 (1993); Burton and Barbas, Human Antibodies from combinatorial libraries. Id., pp 65-82).

The anti-HMW-MAA antibodies may be administered by any suitable means, including parenteral, subcutaneous, intraperitoneal, intrapulmonary, and intranasal. Parenteral infusions include intramuscular, intravenous, intraarterial, intraperitoneal, intralymphatic or subcutaneous administration. In addition, the antibodies may be administered by pulse infusion, e.g., with declining doses of the antibody.

One may also administer other compounds, such as chemotherapeutic agents, immunosuppressive agents and/or cytokines with the anti-HMW-MAA antibodies. The combined administration can include co-administration, using separate formulations or a single pharmaceutical formulation, and can also include consecutive administration in either order, wherein preferably there is a time period while both (or all) active agents simultaneously exert their biological activities.

Therapeutic formulations comprising anti-HMW-MAA antibodies may be prepared by mixing with known pharmaceutically acceptable carriers, excipients or stabilizers. It will be recognized by one of skill in the art that the form and character of the pharmaceutically acceptable carrier or diluent is dictated by the amount of active ingredient with which it is to be combined, the route of administration and other well-known variables, such as the size of the individual and the stage of the disease.

The following illustrative examples are provided to further describe, but not to limit the invention.

### EXAMPLE 1

This Example demonstrates HMW-MAA expression by a subpopulation of breast carcinoma stem cells in breast carcinoma stem cell lines.

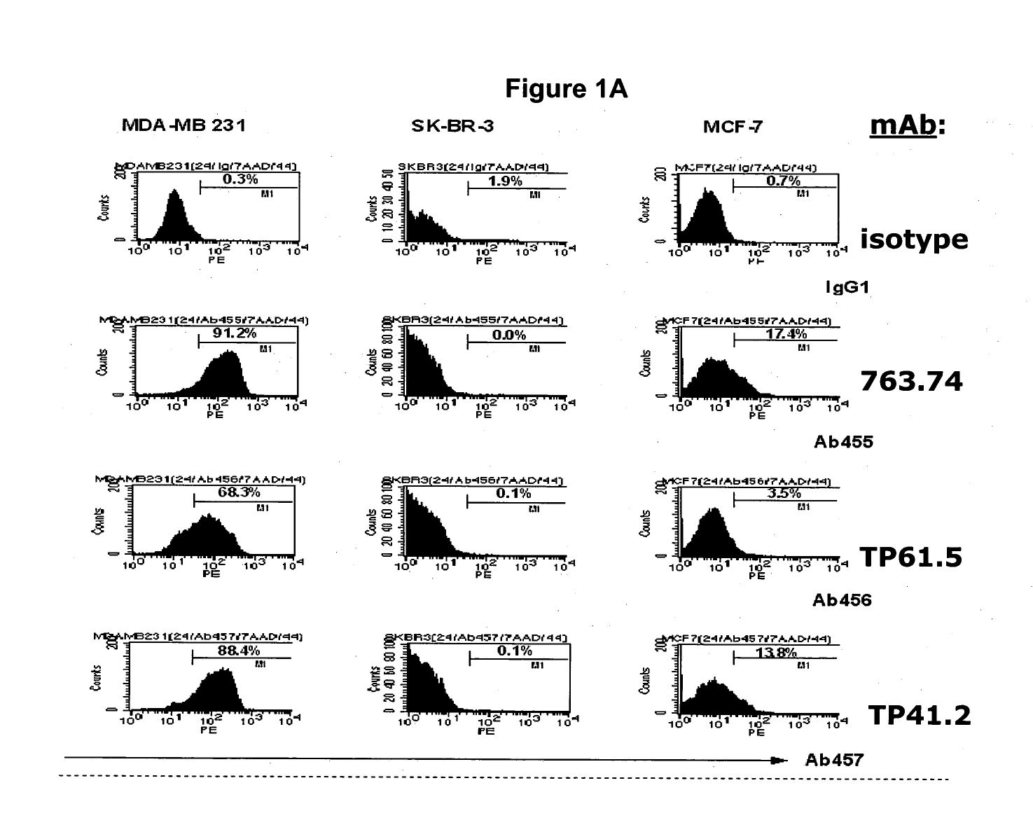

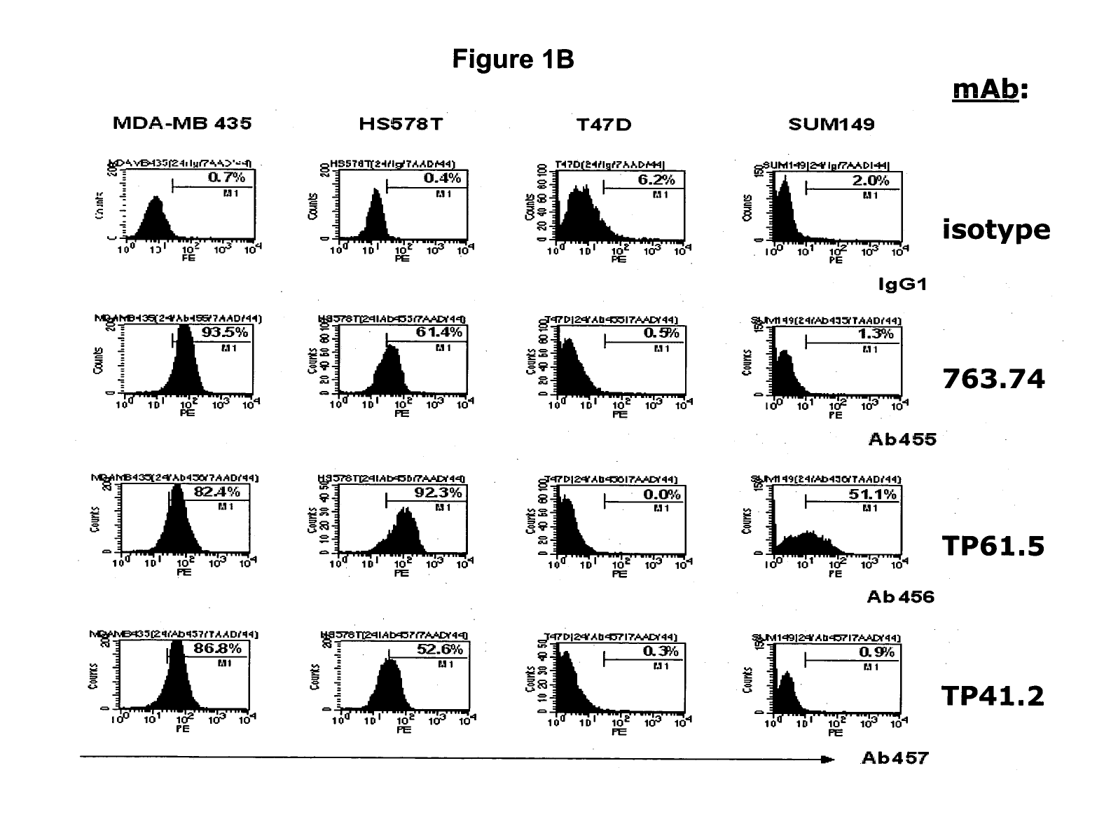

Staining of seven human breast carcinoma cell lines (FIGS. 1A and 1B) with the HMW-MAA-specific mAb 763.74, TP61.5 and VF1-TP41.2 demonstrates that at least 80% of the CD44⁺, CD24 lo cells were stained by HMW-MAA-specific mAb in the cell lines MDA-MB-435, about 70 and 50% in the cell lines MDA-MB-231 and HS578T, respectively, and less than 4% in the cell line MCF-7 and SUM-149. It is noteworthy that the percentage of CD44⁺, CD24 lo cells stained by the three HMW-MAA-specific mAb is stable across multiple cell culture passages, which indicates that the expression of HMW-MAA by breast carcinoma stem cells is a stable characteristic.

### EXAMPLE 2

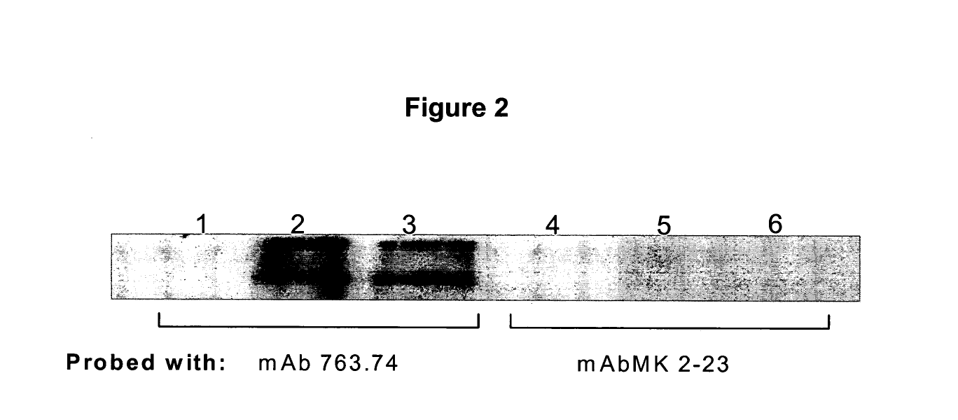

This Example demonstrates the molecular profile of HMW-MAA expressed by breast carcinoma stem cells. To characterize the molecular basis of the staining of breast carcinoma stem cells by HMW-MAA-specific mAb, a lysate of the human breast carcinoma cell line MDA-MB-435 was tested with mAb 763.74 in Western blotting. Specifically, and as shown in FIG. 2, a lysate from CD44⁺CD24^{lo} breast carcinoma cells MDA-MB-435 was separated by 8% SDS-polyacrylamide gel for immunoblot analysis with HMW-MAA-specific mAb 763.74 (lane 3) and isotype control mAb MK2-23 (lane 6). Human melanoma cells M14, which do not express HMW-MAA (lanes 1 and 4), and M14/HMW cells, which express HMW-MAA following HMW-MAA cDNA transfection (lanes 2 and 5), were used as controls. The two characteristic components of the HMW-MAA were identified as depicted in FIG. 2.

### EXAMPLE 3

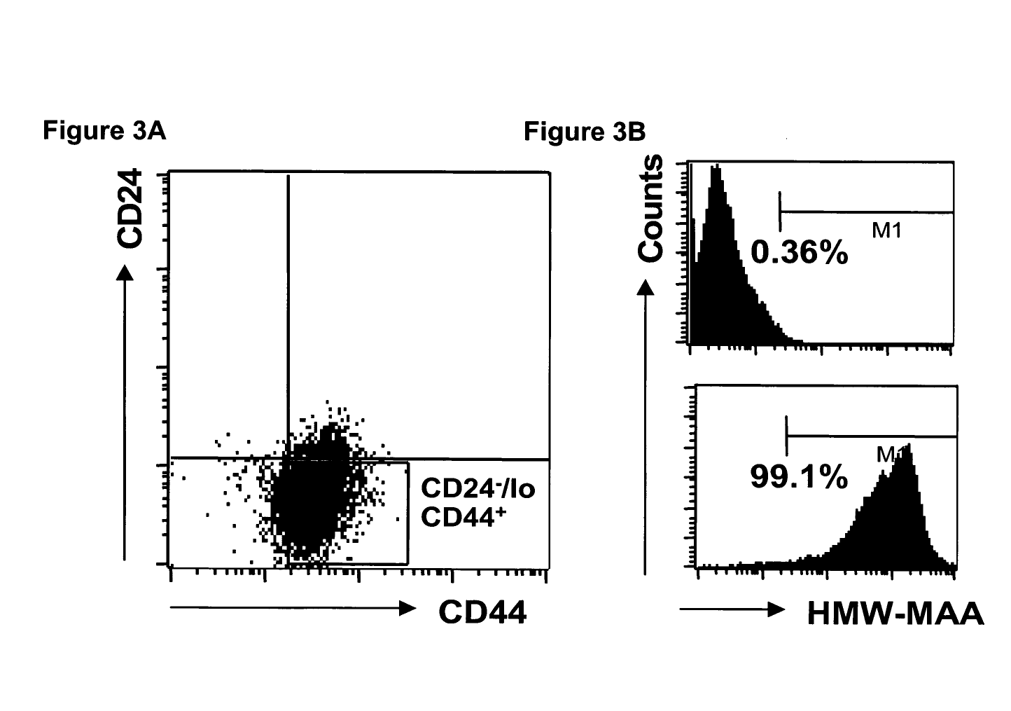

This Example demonstrates HMW-MAA expression by CD44+/CD24−/low breast carcinoma stem cells in the human breast cancer cell line MDA-MB-435s. As depicted in FIG. 3A, staining of MDA-MB-435s cells with CD24−,CD44-specific mAbs showed that >80% of cells are CD44+/CD24−/low breast carcinoma stem cells as indicated. As shown in FIG. 3B, staining of CD44+/CD24−/low putative breast carcinoma stem cells with HMW-MAA-specific mAb 225.28 (bottom panel) and with an isotype control mAb (top panel) showed that 99.1% of CSC are HMW-MAA positive. Thus, a human breast cancer stem cell line is demonstrated to express HMW-MAA.

### EXAMPLE 4

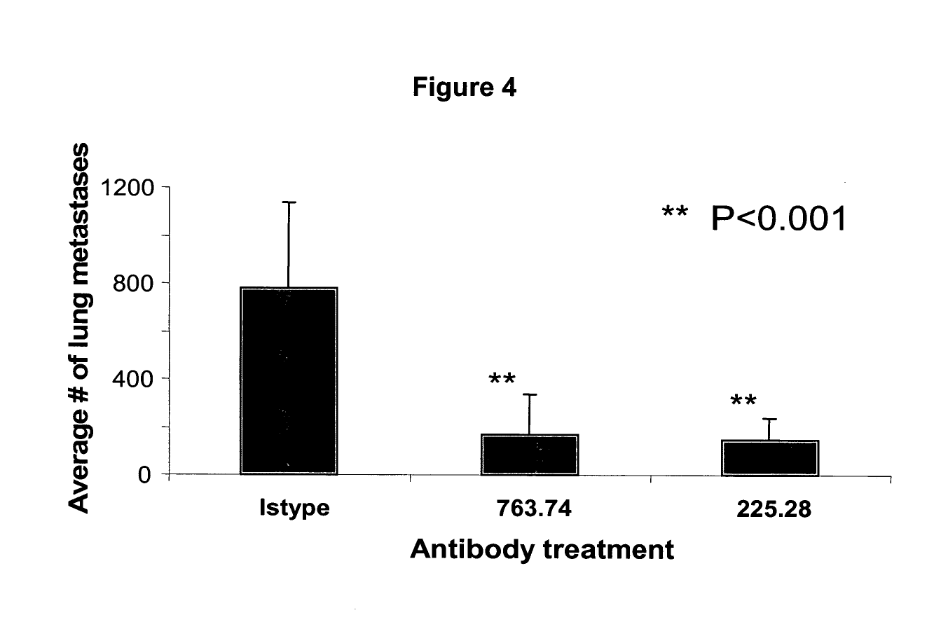

This Example demonstrates inhibition by HMW-MAA-specific mAb 763.74 and 225.28 of human breast carcinoma stem cell (MDA-MB-435s) lung metastases in SCID mice. Results are presented in FIG. 4. To obtain the data shown in FIG. 4, human breast cancer cell MDA-MB-435s (2×106) were injected i.v. into each SCID mouse on day 0. Subsequently, all tumor bearing mice were randomized into three groups (5/group). Starting on day 3, one of the groups was injected i.p. with HMW-MAA-specific mAb 763.74 and one with HMW-MAA-specific mAb 225.28 (100 μg/mouse) twice weekly for a total of 9 injections. The third group of mice was injected with an isotype control antibody. On day 34, all mice were euthanized and lung metastatic nodules were counted. Differences between HMW-MAA-specific mAb treated groups and isotype control antibody treated group were significant (p<0.001).

Thus, this Example demonstrates that administration of either of two distinct HMW-MAA-specific mAbs can inhibit metastasis from tumors produced in an animal model by inoculation with human breast carcinoma stem cells that express HMW-MAA, while administration of an isotyped control mAb that does not bind to HMW-MAA is ineffective in inhibiting such metastasis.

### EXAMPLE 5

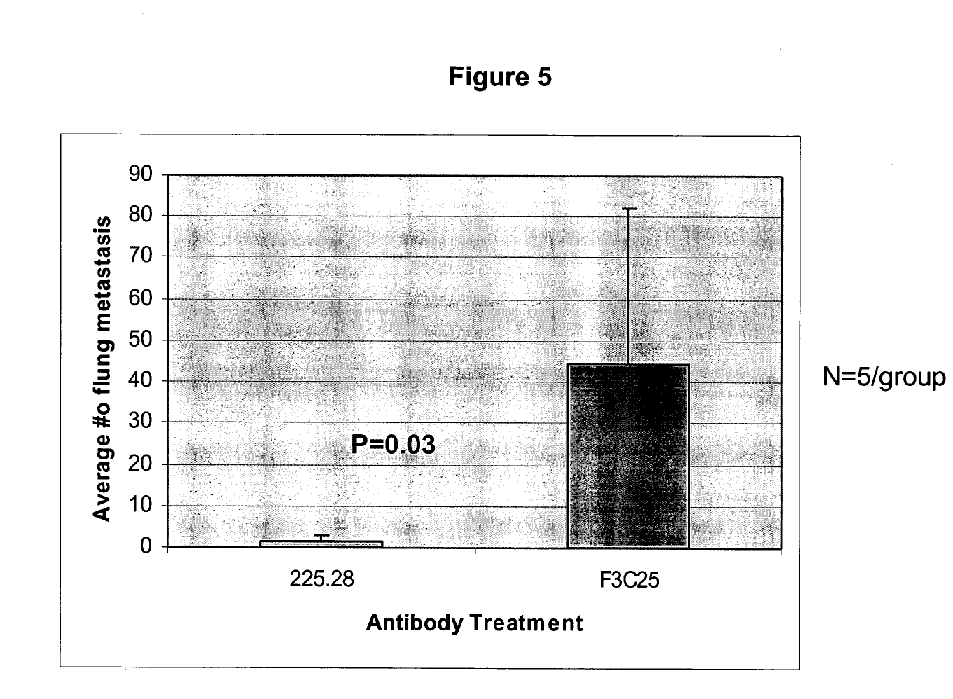

This Example demonstrates inhibition of post-surgery lung metastasis of human breast carcinoma stem cells by use of mAb 225.28. To obtain the data shown in FIG. 5, the following regimen was employed:

Day 0: Mammary fat tumor s.c. inoculation; Day 7: mAb 225.28 treatment with 200 μg/mouse, 2× weekly; Day 71: Surgically remove tumor; Day 103: stop treatment; Day 134: sacrifice mice and collect lungs for metastasis analysis.

As can be seen from FIG. 5, administration of mAb 225.28 results in a statistically significant inhibition of lung metastasis after the removal of a tumor obtained by inoculation of an animal model with human breast carcinoma stem cells that express HMW-MAA.

### EXAMPLE 6

This Example demonstrates inhibition of human breast carcinoma post-surgery reoccurrences by mAbs directed to HMW-MAA.

To obtain the data presented in Table 1, human breast cancer stem cell MDA-MB-435s (2×106) were injected into mammary fat pad of each SCID mouse on day 0. Subsequently, all tumor bearing mice were randomized into three groups (5/group). Starting on day 7, one of the groups was injected i.p. with HMW-MAA-specific mAb 763.74 and one with HMW-MAA-specific mAb 225.28 (200 μg/mouse) twice weekly for a total of 18 injections. The third group of mice was injected with an isotype control antibody. On day 71, all tumors were removed surgically from mice. The treatment with mAb was continued in the same regimen with additional 9 injections. On day 131, all mice were sacrificed, local tumor reoccurrences and lung matastases were detected and analysed.

TABLE 1

| | mAb | | |
|---|---|---|---|
| | 225.28 | F3C25 | 763.74 |
| # of tumor | 0/5 | 3/5 | 1/5 |
| reoccurrences/group | | (1 dead) | |

As can be seen from Table 1, administration of either of two distinct HMW-MAA-specific mAbs results in inhibition of the recurrence of tumors obtained by inoculation of an animal model with human breast carcinoma stem cells that express HMW-MAA, while an isotype control (F3C25) which recognizes an irrelevant antigen does not inhibit such recurrence.

### EXAMPLE 7

This Example demonstrates HMW-MAA expression by subpopulations of breast carcinoma stem cells in pleural exudates from patients with breast cancer.

To obtain the data summarized in Table 2, pleural effusion cells from breast cancer patients were labeled with anti-HMW-MAA mAb (clone 225.28, 763.74, TP41.2, or TP61.5), followed by PE-labeled anti-mouse IgG. After washing, cells were stained with FITC-labeled anti-CD24, PerCP-labeled anti-CD45, APC-labeled anti-CD44, and 7-AAD. Percentages of CD44+CD24− populations in CD45− 7AAD− cells or CD45− 7AAD− HMW-MAA+ cells were analyzed by flow cytometry. Enrichment of CD44+CD24− population by gating at HMW-MAA positive cells was calculated by dividing the percentages of CD44+CD24− cells in CD45− 7AAD− HMW-MAA+ population with that in CD45− 7AAD− population and are shown in parenthesis in each well. The highest fold enrichment is shown at the right column for each patient's sample.

TABLE 2

| | Total cell | | | | | | | | | | | | highest |
|---|---|---|---|---|---|---|---|---|---|---|---|---|---|
| | number | CD44 + CD24− | % of CD44 + CD24− In HMW − MAA+ cells (fold enrichment) | | | | | | | | | | fold |
| patient | (1 × 106) | (%) | mAb 225.28 | | mAb 763.74 | | mAb TP41.2 | | mAb TP61.5 | | Average | | enrichment |
| P4 | 280 | 2.91 | 8.60 | (2.96) | 5.7 | (1.96) | 10.2 | (3.51) | 24.7 | (8.49) | 12.3 | (4.23) | 8.49 |
| P5 | 4170 | 16.3 | 26.8 | (1.64) | 68.9 | (4.23) | 23.7 | (1.45) | 18.3 | (1.12) | 34.4 | (2.11) | 4.23 |
| P6 | 298 | 7.21 | 4.70 | (0.65) | 0.00 | (0.0) | 3.57 | (0.50) | 50.0 | (6.93) | 14.6 | (2.02) | 6.93 |
| P7 | 220 | 19.2 | 35.5 | (1.85) | 95.4 | (4.97) | 61.3 | (3.19) | 75.4 | (3.93) | 66.9 | (3.48) | 4.97 |
| P8 | 360 | 18 | 2.90 | (0.16) | 60.7 | (3.37) | 20.9 | (1.16) | 31.3 | (1.74) | 29.0 | (1.61) | 3.37 |
| P9 | 98 | 3.38 | 22.6 | (6.69) | 40.2 | (11.89) | 38.5 | (11.39) | 35.7 | (10.56) | 34.3 | (10.13) | 11.89 |
| P10 | 1300 | 31.6 | 91.5 | (2.90) | 96.8 | (3.06) | 93.7 | (2.97) | 94.8 | (3.00) | 94.2 | (2.98) | 3.06 |
| P11 | 200 | 13 | 67.4 | (5.18) | 93.3 | (7.18) | 70.3 | (5.41) | 75.5 | (5.81) | 76.6 | (5.89) | 7.18 |
| P12 | 1000 | 4.94 | 13.5 | (2.73) | 96.2 | (19.47) | 90.3 | (18.28) | 67.3 | (13.62) | 66.8 | (13.53) | 19.47 |
| P13 | 2515 | 11.6 | 71.0 | (6.12) | 81.4 | (7.02) | 68.7 | (5.92) | 76.7 | (6.61) | 74.5 | (6.42) | 7.02 |
| P14 | 47 | 12.2 | 8.40 | (0.69) | 91.5 | (7.50) | 49.1 | (4.02) | 32.0 | (2.62) | 45.3 | (3.71) | 7.5 |
| P15 | 58 | 58.7 | 69.3 | (1.18) | 90.3 | (1.54) | nd | | nd | | 79.8 | (1.36) | 1.54 |
| Average | 878.83 | 16.59 | 35.18 | (2.73) | 68.4 | (6.02) | 48.2 | (5.25) | 52.9 | (5.86) | | | 7.14 |
| (fold | | | | | | | | | | | | | |
| enrichment) | | | | | | | | | | | | | |

Thus, this Example demonstrates the presence of breast carcinoma stem cells that express HMW-MAA in human breast cancer patients.

This invention has been described through examples presented above. Routine modifications to the methods and compositions presented herein will be apparent to those skilled in the art and are intended to be within the scope of the claims appended hereto.

We claim:

1. A method for inhibiting the growth of breast carcinoma stem cells in an individual comprising administering to the individual an effective amount of an antibody reactive with High Molecular Weight-Melanoma Associated Antigen (HMW-MAA) or an HMW-MAA reactive fragment thereof, wherein the breast carcinoma stem cells express HMW-MAA+.

2. The method of claim 1, wherein the antibody is a monoclonal antibody.

3. The method of claim 1, wherein the fragment is selected from the group consisting of Fab, Fab′, F(ab′)₂, and Fv.

4. The method of claim 2, wherein the monoclonal antibody is conjugated to an agent selected from the group consisting of toxins and radioactive isotopes.

5. The method of claim 4, wherein the radioactive isotope is selected from the group consisting of, I¹²³, I¹²⁵ I¹²⁴ and I¹³¹.

6. The method of claim 1, wherein the antibody is administered simultaneously or sequentially with a chemotherapeutic agent.

7. The method of claim 1, wherein antibody is administered by a route selected from the group consisting of parenteral, subcutaneous, intraperitoneal, intravenous, intralymphatic and intrapulmonary administration.

8. The method of claim 1, wherein the antibody is administered subsequent to resection of a breast carcinoma.

9. A method for inhibiting metastasis of a breast carcinoma in an individual, wherein the breast carcinoma comprises HMW-MAA+ breast carcinoma stem cells, the method comprising administering to the individual an effective amount of an antibody reactive with HMW-MAA or an HMW-MAA reactive fragment thereof.

10. The method of claim 9, wherein the antibody is a monoclonal antibody.

11. The method of claim 9, wherein the fragment is selected from the group consisting of Fab, Fab′, F(ab′)₂, and Fv.

12. The method of claim 10, wherein the monoclonal antibody is conjugated to an agent selected from the group consisting of toxins and radioactive isotopes.

13. The method of claim 12, wherein the radioactive isotope is selected from the group consisting of, I¹²³, I¹²⁵ I¹²⁴ and I¹³¹.

14. The method of claim 9, wherein the antibody is administered simultaneously or sequentially with a chemotherapeutic agent.

15. The method of claim 9, wherein antibody is administered by a route selected from the group consisting of parenteral, subcutaneous, intraperitoneal, intravenous, intralymphatic and intrapulmonary administration.

16. The method of claim 9, wherein the antibody is administered subsequent to resection of a breast carcinoma.

17. A method for detecting an HMA-MAA+ breast carcinoma stem cell comprising administering to an individual, or contacting a biological sample obtained from the individual with, a combination of antibodies wherein the combination comprises an antibody directed to HMW-MAA and at least one antibody directed to a breast cancer stem cell marker, wherein detecting the binding of both the HMW-MAA antibody and the at least one breast cancer stem cell marker determines the presence of an HMA-MAA+ breast carcinoma stem cell.

18. The method of claim 17, wherein the antibody is a monoclonal antibody.

19. The method of claim 18, wherein the monoclonal antibody is conjugated to a radioactive isotope.

20. The method of claim 19, wherein the antibody directed to a breast cancer stem cell marker is directed to CD44, CD24, and combinations thereof.
